(19) United States
(12) Patent Application Publication
(10) Pub. No.: US 2021/0315721 A1
(43) Pub. Date: Oct. 14, 2021

(54) STENT DELIVERY DEVICE

(71) Applicant: HANGZHOU ENDONOM MEDTECH CO., LTD, Zhejiang (CN)

(72) Inventors: Jianmin Li, Zhejiang (CN); Yongsheng Wang, Zhejiang (CN)

(21) Appl. No.: 17/358,434

(22) Filed: Jun. 25, 2021

Related U.S. Application Data
(63) Continuation of application No. PCT/CN2019/128095, filed on Dec. 24, 2019.

(30) Foreign Application Priority Data
Dec. 28, 2018 (CN) 201811625327.2
Dec. 28, 2018 (CN) 201822243522.0

(51) Int. Cl. A61F 2/966; A61F 2/95

(52) U.S. Cl. CPC A61F 2/966; A61F 2002/9505; A61F 2/9522

(57) ABSTRACT

A stent delivery device for releasing a stent into a vessel. The stent delivery device includes a fixing assembly including a fixing member and a limiting member, wherein one end of the limiting member passes through the stent and abuts against the fixing member to lock the stent; and a releasing assembly including a movable member and a locking member connected to the movable member, wherein the movable member is connected to the other end of the limiting member, and the locking member is slidably connected to the movable member. When the locking member is in a first position, the movable member is fixed relative to the fixing member under the restriction of the locking member. When the locking member slides to a second position, the movable member can drive the limiting member to move away from the fixing member to release the stent.

### REFERENCE TO RELATED APPLICATIONS

This application is a continuation of PCT/CN2019/128095 filed Dec. 24, 2019, the entire contents of which are incorporated herein by reference in its entirety.

### TECHNICAL FIELD

The present application relates to the technical field of medical instruments, and more particularly to a stent delivery device.

### BACKGROUND

Aortic diseases, such as aortic aneurysm and aortic dissection, are one kind of the most pernicious and hardest vascular surgical diseases to treat. Traditional treatment methods, such as laparotomy and prosthetic vessel replacement, have the risks of severe surgical trauma and high fatality rate. In recent years, a minimally invasive and simple interventional operation method has been developed, in which a covered stent is implanted in a lesion site of a vessel, and the covered stent conforms to the inner wall of the vessel to isolate the lesion site of the vessel from the blood flow. The covered stent can not only allow the blood to flow through normally, but also protect the lesion site of the vessel and effectively repair the lesion site of the vessel. However, how to provide a stent delivery device capable of effectively releasing the stent into the lesion site of the vessel to improve the effect of treating vascular diseases has become a technical problem to be solved.

### SUMMARY

The present application provides a stent delivery device capable of effectively releasing a stent into a lesion site of a vessel.

The stent delivery device provided in the present application is configured to release the stent into the vessel, and includes:

a fixing assembly, including a fixing member and a limiting member, wherein one end of the limiting member passes through the stent and abuts against the fixing member so as to lock the stent; and

a releasing assembly, including a movable member and a locking member connected to the movable member, wherein the movable member is connected to the other end of the limiting member, the locking member is slidably connected to the movable member, and wherein when the locking member is in a first position, the movable member is fixed relative to the fixing member under the restriction of the locking member, and when the locking member slides to a second position, the movable member is capable of driving the limiting member to move away from the fixing member so as to release the stent.

The locking member is provided to lock the movable member so as to prevent the movable member from driving the limiting member to release the stent when there is no need to release the stent. The locking member is provided to move to different positions so as to control the locking member to lock or release the movable member. Since the position of the locking member is variable, it may be rapidly determined whether the movable member is in a fastened state or a released state. Further, when the movable member is in the released state, the limiting member may be driven to lock or release the stent, so as to speed up the release of the stent and save the surgical treatment time.

### BRIEF DESCRIPTION OF THE DRAWINGS

In order to illustrate the technical solutions of the embodiments according to the present application more clearly, drawings used in the description of the embodiments according to the present application will be briefly introduced below. It should be appreciated that the drawings described below merely illustrate some embodiments of the present application, and other drawings may be obtained by those skilled in the art without departing from the scope of the drawings.

FIG. 1 is a schematic diagram showing a structure of a stent delivery device according to an embodiment of the present application.

FIG. 2 is a cross-sectional view showing a stent delivery device according to an embodiment of the present application.

FIG. 3 is a partial cross-sectional view showing a proximal end of a stent delivery device according to an embodiment of the present application.

FIG. 4 is a partial cross-sectional view showing a distal end of a stent delivery device according to an embodiment of the present application.

FIG. 5 is a schematic diagram showing a structure of a distal end of a stent delivery device according to an embodiment of the present application.

FIG. 6 is an exploded view of a releasing assembly in a stent delivery device according to an embodiment of the present application.

FIG. 7 is a partial perspective view showing a proximal end of a stent delivery device according to an embodiment of the present application.

FIG. 8 is a cross-sectional view showing an outer sheath sliding assembly in a stent delivery device according to an embodiment of the present application.

FIG. 9 is a partial enlarged cross-sectional view showing a switch in an outer sheath sliding assembly of a stent delivery device according to an embodiment of the present application.

### DESCRIPTION OF THE EMBODIMENTS

The technical solutions of the embodiments of the present application will be clearly and fully described below in combination with accompanying drawings in the embodiments of the present application. For ease of description, “proximal end”, “distal end” and “first direction” are involved, wherein the term “proximal end” refers to one end away from an operating end of the stent delivery device, and the term “distal end” refers to one end close to the operating end of the stent delivery device, and the term “first direction” refers to an extending direction of the stent delivery device.

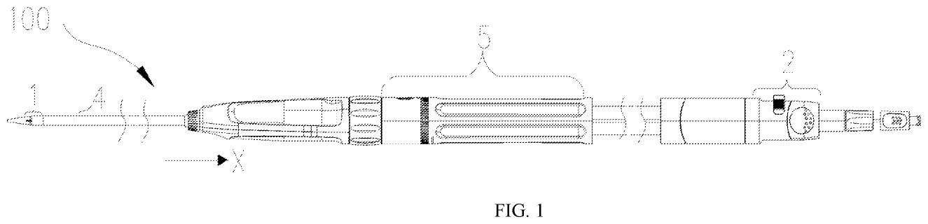

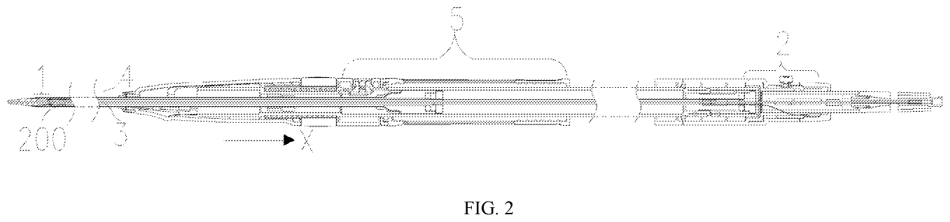

Referring to FIG. 1 and FIG. 2, FIG. 1 shows a stent delivery device 100 according to an embodiment of the present application. The stent delivery device 100 is configured to release the stent 200 into a lesion site of a vessel. The stent 200 includes a tubular rigid wire frame and a polymer membrane fixed on a surface of the tubular rigid wire frame. The tubular rigid wire frame may be obtained by forming an elastic rigid wire bended in a Z-shaped manner into a plurality of rings, and the stent 200 with a membrane is formed by stitching or bonding the plurality of rings with the polymer membrane. Due to the elasticity of the rigid wire, the tubular rigid wire frame is capable of being compressed (here it mainly refers to being compressed radially, but it should be understood that the tubular rigid wire frame will be extended in its axial direction accordingly after being radially compressed). After the external force is removed, the tubular rigid wire frame is able to expand or deploy upon its elasticity to restore its original shape. During use, the stent 200 is loaded into the stent delivery device 100 after being radially compressed and delivered to the lesion site of the vessel by the stent delivery device 100 and then released. Due to the elasticity of the tubular rigid wire frame, the stent 200 automatically returns to the radially expanded state and closely conforms to an inner wall of the vessel to isolate the lesion site of the vessel from the blood flow, thereby achieving the treatment.

With reference to FIG. 1 and FIG. 2, the stent delivery device 100 includes a fixing assembly 1 and a releasing assembly 2. The releasing assembly 2 and the fixing assembly 1 are arranged along a first direction X. The fixing assembly 1 is located at a proximal end of the stent delivery device 100, and the releasing assembly 2 is located at a distal end of the stent delivery device 100, wherein the fixing assembly 1 is configured to secure the stent 200, and the releasing assembly 2 is configured to manipulate the fixing assembly 1 so as to release the stent 200.

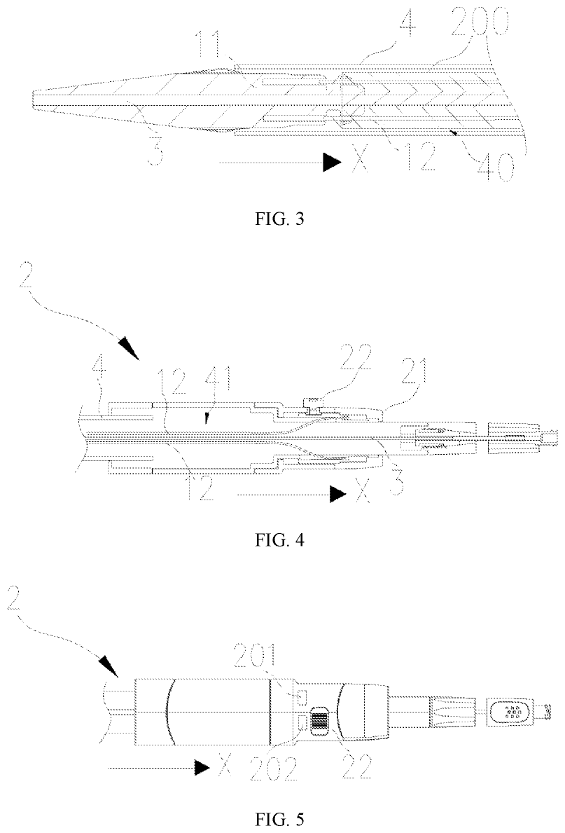

With reference to FIG. 3, the fixing assembly 1 includes a fixing member 11 and a limiting member 12. One end of the limiting member 12 extends through the stent 200 and is slidably connected to the fixing member 11 to lock the stent 200.

Particularly, with reference to FIG. 3, both the stent 200 and the limiting member 12 extend along the first direction X. The proximal end of the stent 200 is provided with a hollowing portion. One end of the limiting member 12 (a proximal end of the limiting member 12) extends through the hollowing portion of the stent 200 and is slidably connected to the fixing member 11, so that the proximal end of the stent 200 is tightened and fixed. In other words, the stent 200 is mounted around the limiting member 12. The distal end of the stent 200 is adjacent to an operating end of the stent delivery device 100, and the distal end of the stent 200 is tightened and restrained by other position of the stent delivery device 100, such that the stent 200 is wholly tightened and restrained to the stent delivery device 100, which brings the convenience for delivering the stent 200 into the vessel with the stent delivery device 100.

With reference to FIG. 4, the releasing assembly 2 includes a movable member 21 and a locking member 22 connected to the movable member 21. The movable member 21 is connected to the other end of the limiting member 12 (a distal end of the limiting member 12). The movable member 21 is configured to drive the limiting member 12 to move away from or close to the fixing member 11 in the first direction X, so that the stent 200 is released or locked by the limiting member 12. The locking member 22 is configured to lock the movable member 21, to prevent the movable member 21 from moving in the first direction X, and thus prevent the limiting member 12 from releasing the stent 200 due to unintentional touch and other reasons when there is no need to release the stent 200 which results in an incorrect position where the stent 200 is released and further incapability of treating the vascular diseases.

With reference to FIG. 4, the locking member 22 is slidably connected to the movable member 21. When the locking member 22 is located at a first position, the movable member 21 is fixed relative to the fixing member 11 under the restriction of the locking member 22. In other words, when the locking member 22 is in the first position, the movable member 21 is locked, so that the movable member 21 cannot drive the limiting member 12 to move in the first direction X. At that time, the limiting member 12 is connected to the fixing member 11. When the locking member 22 slides to a second position, the movable member 21 can drive the limiting member 12 to move away from the fixing member 11 to release the stent 200. In other words, when the locking member 22 is located at the second position, the movable member 21 can drive the limiting member 12 to move in the first direction X, so that the limiting member 12 can moves out from the fixing member and then out from the hollowing portion of the stent 200. When the limiting member 12 moves to a certain position, it is disengaged from the proximal end of the stent 200. As a result, the stent 200 can be released into the vessel. After the stent 200 is released, it expands radially due to its elasticity.

The locking member 22 is provided to lock the movable member 21, so as to prevent the movable member 21 from driving the limiting member 12 to release the stent 200 when there is no need to release the stent 200. The locking member 22 is configured such that it can move to different positions, to fasten or release the movable member 21 by controlling the locking member 22. Since the position of the locking member 22 is changeable, it may be rapidly determined that the movable member 21 is in a locked state or a released state. Further, when the movable member 21 is in the released state, the limiting member 12 may be driven to lock or release the stent 200, to speed up the release of the stent 200 and save the surgical treatment time.

In a possible embodiment, with reference to FIG. 5, an outer surface of the releasing assembly 2 is provided with a first marker 201. When the locking member 22 is located at the first position, the locking member 22 is aligned with the first marker 201, and the locking member 22 locks the movable member 21 and the limiting member 12 locks the stent 200 if the stent 200 is provided on the limiting member 12. When the locking member 22 is located at the second position, the locking member 22 is offset from the first marker 201, the movable member 21 is released by the locking member 22, and the movable member 21 is capable of driving the limiting member 12 to move to release the stent 200 therefrom.

The first marker 201 may correspond to a state in which the locking member 22 locks the movable member 21. The first marker 201 may be a protrusion or groove provided on the outer surface of the releasing assembly 2, or a marker patch or the like attached to the outer surface of the releasing assembly 2. Certainly, in other embodiments, the first marker 201 may be a status indicator lamp, a buzzer, or the like.

Due to the first marker 201 provided on the outer surface of the releasing assembly 2, which is readily visible by an operator, it is convenient for the operator to quickly and directly determine whether the movable member 21 is in the locked state or the released state. Accordingly, the intuitiveness of determination is improved, the operation is simplified, and the time is saved.

Further, with reference to FIG. 5, the outer surface of the releasing assembly 2 is further provided with a second marker 202 spaced apart from the first marker 201. When the locking member 22 is located at the second position, the locking member 22 is aligned with the second marker 202.

The first marker 201 and the second marker 202 may be spaced apart and provided on an outer peripheral surface of the releasing assembly 2. The first marker 201 is adjacent to and aligned with the first position, and the second marker 202 is adjacent to and aligned with the second position.

The second marker 202 may correspond to a state in which the fastening member 22 releases the movable member 21. Similarly, the second marker 202 may be a protrusion or groove provided on the outer surface of the releasing assembly 2, or a marker patch attached to the outer surface of the releasing assembly 2. It may further be a state indicator lamp, a buzzer, or the like.

Due to the first marker 201 and the second marker 202 provided on the outer surface of the releasing assembly 2, which are readily visible by the operator, it is convenient for the operator to quickly and directly determine whether the movable member 21 is in the locked state or the released state. Accordingly, the intuitiveness of determination is improved, the operation is simplified, and the time is saved.

With reference to FIG. 2, the movable member 21 has a central axis. The center axis runs along the direction X. The fixing member 11, the limiting member 12 and the movable member 21 are arranged along an extending direction of the central axis.

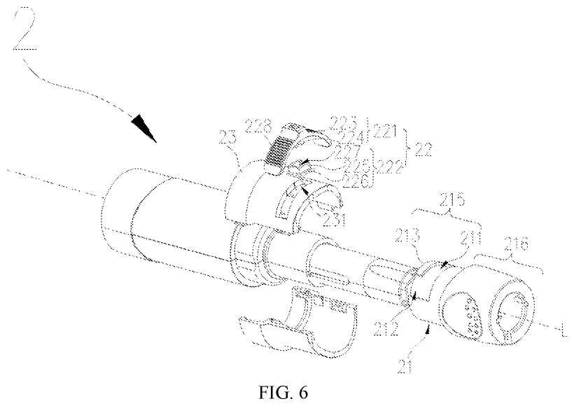

In a possible embodiment, with reference to FIG. 6, an outer peripheral surface of the movable member 21 is provided with a first sliding groove 211 and a second sliding groove 212 communicating end to end. The first sliding groove 211 extends along a circumferential direction of the movable member 21. The second sliding groove 212 extends along the central axis L. One end of the locking member 22 can slide along the first sliding groove 211 and the second sliding groove 212.

The movable member 21 may be of a cylindrical shape, with an axial direction thereof as the first direction X. Certainly, in other embodiments, the movable member 21 may also be of a square tubular shape or other tubular shape.

Due to the first sliding groove 211 and the second sliding groove 212 provided in the outer circumferential surface of the movable member 21, with the first sliding groove 211 extending along the circumferential direction of the movable member 21, and the second sliding groove 212 extending along the first direction X, when the locking member 22 is located in the first sliding groove 211, the locking member 22 locks the movable member 21 in the first direction X to prevent the movable member 21 from moving in the first direction X, and the limiting member 12 abuts against the fixing member 11. If the limiting member 12 extends through the stent 200, the limiting member 12 fixes the stent 200 on the stent delivery device 100. When the locking member 22 slides to the second sliding groove 212, the locking member 22 releases the movable member 21, so that the movable member 21 is capable of moving in the first direction X, and in turn the movable member 21 is capable of driving the limiting member 12 to move away from the fixing member 11 in the first direction X, so as to release the stent 200 on the limiting member 12, or the movable member 21 is capable of driving the limiting member 12 to move close to the fixing member 11 along the first direction X, so as to make the limiting member 12 return to an initial position after the stent 200 is released.

Further, the first position and the second position are located at the first sliding groove 211 and the second sliding groove 212, respectively. The first position may be any position in the first sliding groove 211, and the second position may be any position in the second sliding groove 212. That is, when the locking member 22 is located in the first sliding groove 211, the movable member 21 is fixed relative to the fixing member 11 under the restriction of the locking member 22. When the locking member 22 slides to the second sliding groove 212, the movable member 21 is capable of driving the limiting member 12 to move away from the fixing member 11 so as to release the stent 200.

Further, with reference to FIG. 6, the movable member 21 has a first end surface 213, through which the second sliding groove 212 extends. When the movable member 21 moves from the proximal end to the distal end along the direction X and moves away from the fixing member 11, the locking member 22 is capable of sliding out of the second sliding groove 212 until it is disengaged from the movable member 21. It should be understood that the first end surface 213 is adjacent to the fixing member 11.

Due to the second sliding groove 212 extending through the first end surface 213, the locking member 22 is capable of sliding out of the second sliding groove 212, and the movable member 21 is then disengaged from the locking member 22. The movable member 21 is controlled to drive the limiting member 12 along the first direction X, so that the limiting member 12 may be quickly separated from the fixing member 11 so as to quickly release the stent 200. Therefore, the stent 200 is released at an accurate position and the surgical time is reduced. Moreover, the movable member 21 disengaged from the locking member 22 is capable of moving freely along an appropriate course, to drive the limiting member 12 to move an appropriate distance, such that the limiting member 12 is capable of releasing the stent 200 without moving too far from the fixing member 11 which results in difficulty of returning to the initial position.

With reference to FIG. 6, the releasing assembly 2 further includes a first sleeve 23. The first sleeve 23 is mounted around a periphery of the movable member 21. The first sleeve 23 is provided with a limiting hole 231. The locking member 22 is arranged on the first sleeve 23. The locking member 22 is slidably connected to the movable member 21 through the limiting hole 231.

It should be understood that the central axis L of the first sleeve 23 runs along the first direction X. The first sleeve 23 can restrict the locking member 22 in a radial direction, so that the locking member 22 may be slidably connected to the movable member 21.

In a possible embodiment, the limiting hole 231 is an elongated hole. The limiting hole 231 may extend along a circumferential direction of the first sleeve 23. When the locking member 22 slides, the first sleeve 23 is fixed relative to the fixing member 11, and the locking member 22 can slide in the limiting hole 231. In other embodiments, the locking member 22 may be fixed on the first sleeve 23. When the locking member 22 slides, the locking member 22 and the first sleeve 23 rotate together around the first direction X, to allow one end of the locking member 22 to be slidably connected to the movable member 21.

Further, with reference to FIG. 6, the limiting hole 231 extends along the circumferential direction of the first sleeve 23. The locking member 22 can slide from one end of the first sliding groove 211 to the other end of the first sliding groove 211 along the limiting hole 231. An inner wall of the limiting hole 231 restricts the locking member 22 to be fixed relative to the first sleeve 23 in the direction of the central axis.

The limiting hole 231 which is configured as an elongated hole extending in the circumferential direction provides a sliding space for the locking member 22, so that the locking member 22 is capable of sliding along the circumferential direction of the first sleeve 23 with the locking member 22 being restricted to be axially fixed relative to the first sleeve 23. Therefore, the position of the locking member 22 is changeable, which facilitates quick recognition of the movable member 21 in the locked state or the released state, and thus increase the speed of releasing the stent 200.

Further, when the locking member 22 slides out of the second sliding groove 212, the movable member 21 is disengaged from the first sleeve 23 and the locking member 22, and the limiting member 12 moves away from the fixing member 11 when being driven by the movable member 21, and then releases the stent 200 to achieve the treatment of the vascular disease.

In a possible embodiment, with reference to FIG. 6, the movable member 21 includes a connecting portion 215 and an operating portion 216 connected to each other. The connecting portion 215 is provided with the first sliding groove 211 and the second sliding groove 212. The first sleeve 23 is mounted around an outer peripheral surface of the connecting portion 215. The connecting portion 215 is connected to the limiting member 12. The operating portion 216 is arranged apart from the first sleeve 23, and is configured to move under an external force, and drive the connecting portion 215 and the limiting member 12 to move away from or close to the fixing member 11.

With reference to FIG. 6, the operating portion 216 may be a handheld structure. When the movable member 21 is released from the fastener 22, an operator holds the operating portion 216 with his or her hand and pulls the operating portion 216 towards a distal end of the stent delivery device 100. The operating portion 216 drives the connecting portion 215 to approach to the distal end of the stent delivery device 100, and thus the limiting member 12 approaches to the distal end of the stent delivery device 100 under the action of the connecting portion 215, so that the limiting member 12 gradually moves away from the fixing member 11, and thus the stent 200 is quickly released at the lesion site of the vessel.

In a possible embodiment, with reference to FIG. 6, the locking member 22 includes a toggle portion 221 and a sliding portion 222 connected to each other. The toggle portion 221 abuts against an outer peripheral surface of the first sleeve 23. The sliding portion 222 passes through the limiting hole 231 and abuts against an inner peripheral surface of the first sleeve 23. The toggle portion 221 is configured to slide relative to the first sleeve 23 under an external force, and drive the sliding portion 222 to slide in the first sliding groove 211 and the second sliding groove 212 through the limiting hole 231 of the first sleeve 23.

The sliding portion 222 may slide along the limiting hole 231 by sliding the toggle portion 221 by the operator, that is, the operator may make the locking member 22 lock or release the movable member 21 by operating the toggle portion 221. The operation is simple and convenient, with high feasibility, and quick implementation, and the efficiency of releasing the stent 200 is improved.

Further, the toggle portion 221 is detachably connected to the sliding portion 222, so that the locking member 22 may be conveniently and quickly mounted on the first sleeve 23.

In a possible embodiment, with reference to FIG. 6, the toggle portion 221 is snap-fit with the sliding portion 222. The toggle portion 221 includes a cover plate 223 and a hook 224 arranged on the cover plate 223. The cover plate 223 abuts against the outer surface of the first sleeve 23. The sliding portion 222 is provided with a first sliding portion 225 and a second sliding portion 226 connected to each other, with a through hole 227 extending through the first sliding portion 225 and the second sliding portion 226. The first sliding portion 225 is slidably engaged in the limiting hole 231 of the first sleeve 23, and the second sliding portion 226 is slidably engaged in the first sliding groove 211 and the second sliding groove 212 of the movable member 21. The first sliding portion 225 may be configured to slide along the limiting hole 231 of the first sleeve 23 when being driven by the toggle portion 221. In one embodiment, the first sliding portion 225 is configured to be at least partially accommodated in the limiting hole 231 of the first sleeve 23. The second sliding portion 226 is configured to slide in the first sliding groove 211 and the second sliding groove 211 of the movable member 21. The hook 224 is engaged with the second sliding portion 226 through the through hole 227, so that one end of the first sliding portion 225 abuts against the cover plate 223.

In other embodiments, the toggle portion 221 may be threadedly or magnetically connected to the sliding portion 222.

Further, with reference to FIG. 6, textures 228 is provided on an outer surface of the cover plate 223, such that when the operator manually toggles the cover plate 223, the textures 228 serves to avoid skidding.

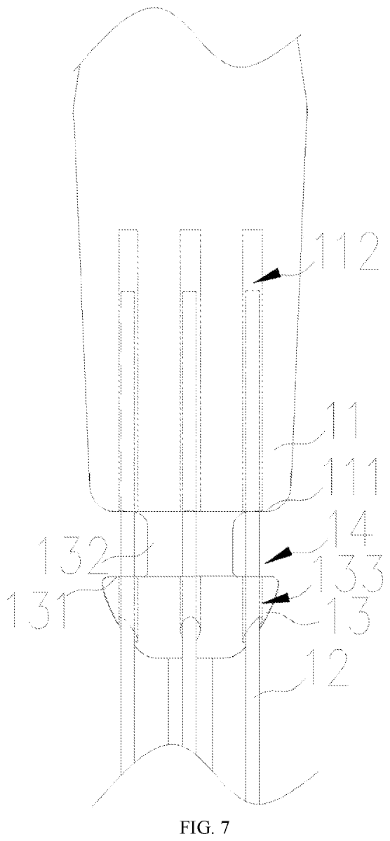

In combination with any one of the foregoing embodiments, in a possible embodiment, with reference to FIG. 7, the fixing assembly 1 further includes a guide member 13. The guide member 13 is closer to an operating end of the stent delivery device 100 relative to the fixing member 11. An accommodating space 14 is defined between the guide member 13 and the fixing member 11. The accommodating space 14 is configured to accommodate an end portion of the stent 200. One end of the limiting member 12 extends through the guide member 13 and extends into the accommodating space 14 so as to pass through the end portion of the stent 200 and abut against the fixing member 11.

With reference to FIG. 7, the guide member 13 may be fixedly connected to the fixing member 11. The fixing member 11 is provided with a first surface 111 directly facing the guide member 13. The guide member 13 is provided with a second surface 131 directly facing the fixing member 11. A protrusion 132 may be provided on the second surface 131. The protrusion 132 may be connected to the first surface 111. A radial size of the protrusion 132 may be smaller than that of the first surface 111 and the second surface 131, that is, the protrusion 132 is sunken in the radial direction relative to the fixing member 11 and the guide member 13. The first surface 111, the second surface 131 and the outer peripheral surface of the protrusion 132 cooperatively define the accommodating space 14.

Further, the guide member 13 may be integrally formed with the fixing member 11 as one piece, that is, one end of the protrusion 132 is integrally formed with the guide member 13 and the other end of the protrusion 132 is integrally formed with the fixing member 11.

As described above, the stent 200 has a tubular shape, with an end having the hollowing portion. The fixing member 11 and the guide member 13 are provided in the fixing assembly 1. The limiting member 12 extends through the inner cavity of the stent 200. The end of the stent 200 which has the hollowing portion converges radially and inwardly, such that the limiting member 12 can extend through the hollowing portion, and be slidably connected with the fixing member 11, as a result, the hollowing portion at the end of the stent 200 is locked by cooperation of the limiting member 12, the first surface 111 and the second surface 131, and thus the stent 200 is axially limited. The guide member 13 provides guiding for the limiting member 12, thus the limiting member 12 can move away from or close to the fixing member 11 in the first direction X. When the limiting member 12 moves away from the fixing member 11, the stent 200 would not move with the limiting member 12, and thus the limiting member can be withdrawn from the hollowing portion of the stent 200, so that the stent 200 can be released.

Further, with reference to FIG. 7, the first surface 111 is provided with a first hole 112. The first hole 112 may be a blind hole. The first hole 112 extends along the first direction X. When the locking member 22 is in the first position or the second position, one end of the limiting member 12 extends into or exits from the first hole 112 when being driven by the movable member 21. By providing the first hole 112 in the fixing member 11, the limiting member 12 is capable of extending into the first hole 112 to achieve an engagement between the fixing member 11 and the limiting member 12, which facilitates the locking of the stent 2 by the limiting member 12 to prevent the stent 200 from being easily released.

Further, with reference to FIG. 7, the guide member 13 is provided with a second hole 133. The second hole 133 may extend through the guide member 13 along the first direction X. The second hole 133 is aligned with the first hole 112. The limiting member 12 extends through the second hole 133 and is slidably connected to the guide member 13.

A proximal end of the limiting member 12 may extend into the first hole 112 through the second hole 133. When the movable member 21 drives the limiting member 12 to move, the limiting member 12 slides along the second hole 133 to extend into or move out of the first hole 112, thereby locking or releasing the stent 200.

With the second hole 133 provided in the guide member 13, which serves to guide the limiting member 12, and the second hole 133 being aligned with the first hole 112, the limiting member 12 can accurately extend into the first hole 112.

When the limiting member 12 moves away from the fixing member 11 when being driven by the movable member 21, an end portion of the limiting member 12 may be retracted into the second hole 133, and the stent 200 is released from the limiting member 12 due to the blockage of the guide member 13. The guide member 13 serves to facilitate the release of the stent 200 from the limiting member 12.

In a possible embodiment, with reference to FIG. 7, A plurality of the first holes 112 are provided. The plurality of first holes 112 are arranged along a circle. The second holes 133 has a number and an arrangement manner the same as those of the first holes 112. Each first hole 112 is corresponding to one second hole 133. A plurality of the limiting members 12 are provided, and the plurality of limiting members 12 is arranged in a tubular shape. Each of the limiting members 12 extends through one of the second holes 133 and is aligned with or inserted into one of the first holes 112.

With reference to FIG. 3 and FIG. 7, the stent 200 may be tubular. The proximal end of the stent 200 is provided with a plurality of hollowing portions, and the plurality of limiting members 12 respectively passes through the plurality of hollowing portions and insert into the first holes 112 of the fixing member 11, so that the proximal end of the stent 200 is tightened and fixed on the limiting member 12 and thus the end of the stent 200 is fixedly connected relative to the fixing member 11.

The plurality of limiting members 12 are provided to fix the stent 200 along the circumferential direction, so that the proximal end of the stent 200 is tightened and the stent 200 is maintained in a tube shape. The firmness of the stent 200 fixed on the fixing member 11 may be improved.

In combination with any of the foregoing embodiments, in a possible embodiment, with reference to FIG. 3, the stent delivery device 100 further includes an inner core 3. The fixing member 11 and the movable member 21 are mounted around an outer periphery of the inner core 3. The fixing member 11 is fixed relative to the inner core 3. The movable member 21 is slidably connected to the inner core 3.

The inner core 3 may extend along the first direction X. The fixing member 11, the guide member 13, the locking member 22 and the movable member 21 are mounted around the outer periphery of the inner core 3 in sequence.

It should be understood that the inner core 3 may be of a hollow tubular shape, which can conduct a liquid into the vessel.

By providing the inner core 3, the inner core 3 is able to connect the fixing member 11, the guide member part 13, the locking member 22 with the movable member 21 together. Moreover, the inner core 3 also serves to guide the sliding of the movable member 21.

Further, with reference to FIG. 3, the stent delivery device 100 further includes an outer sheath 4. The outer sheath 4 is mounted around the outer periphery of the inner core 3. An accommodating cavity 40 is defined between the outer sheath 4 and the inner core 3. The stent 200 is compressed within the accommodating cavity 40.

The outer sheath 4 and the inner core 3 are provided to define the accommodating cavity 40. Since the accommodating cavity 40 is in an annular shape, the tubular stent 200 may be tightened within the accommodating cavity 40 in a tubular shape. On the one hand, a space within the stent delivery device 100 is fully utilized. On the other hand, the stent 200 is delivered after being tightened, which can reduce the profile of the stent delivery device 100, as a result, the interference of the stent delivery device 100 to the vessel is reduced. Moreover, the stent 200 is delivered in a tubular shape. The stent 200 is capable of easily returning to its original shape after being released, to achieve the treatment at the lesion site of the vessel.

In a possible embodiment, the inner core 3 and the outer sheath 4 are made of flexible materials, which may improve the flexibility of the stent delivery device 100, so that the stent delivery device 100 is able to deliver the stent 200 to a curved vessel, applications of the stent delivery device 100 are increased.

The limiting member 12 may be arranged within the accommodating cavity 40. The stent 200 is arranged between the limiting member 12 and the outer sheath 4.

In case that a plurality of limiting members 12 are provided, the plurality of limiting members 12 are annularly arranged within the accommodating cavity 40. The distal end of the stent 200 is mounted around the outer peripheries of limiting members 12.

As the limiting member 12 is accommodated between the inner core 3 and the outer sheath 4, the limiting member 12 is arranged within the stent 200 with its proximal end being capable of disengaged from the hollowing portion of the stent, and the limiting member 12 would not affect the release of the distal end of the stent 200 after the proximal end of the limiting member 12 is disengaged from the hollowing portion of the stent 200.

Further, the limiting member 12 is made of a flexible material, which further increases the flexibility of the stent delivery device 100, so that the stent delivery device 100 can deliver the stent 200 into the curved vessel, and thus the applications of the stent delivery device 100 are increased.

The limiting elements 12 may be separately arranged within the accommodating cavity 40. When the stent delivery device 100 enters a vessel which extends curvedly, the limiting elements 12 is able to be bent with the inner core 3 and the outer sheath 4, such that the stent delivery device 100 can deliver the stent 200 to the curved vessel.

Further, with reference to FIG. 3, one end of the outer sheath 4 abuts against the fixing member 11. The outer sheath 4 can slide relative to the inner core 3. When the outer sheath 4 moves away from the fixing member 11, a portion of the stent 200 which exposes from the outer sheath 4 expands under the action of its own tension.

One end of the outer sheath 4 abuts against the fixing member 11 to enclose the limiting member 12, the stent 200, and the guide member 13 within the outer sheath 4, so as to prevent the stent 200 from being affected by the outside.

When the outer sheath 4 moves away from the fixing member 11, a portion of the proximal end of the stent 200 which exposes from the outer sheath 4 expands as the restraint of the outer sheath 4 is removed. However, as the limiting member 12 extends through the hollowing portion of the stent 200 and the end of the stent 200 having the hollowing portion is still restricted radially by the limiting member 12, the stent 200 cannot be completely released even though the restriction by the outer sheath 4 is removed. This may be advantageous that it is possible to adjust the axial position of the stent 200 at that time as the stent 200 is not completely expanded yet, and the stent 200 would not exert a large force to the inner wall of the blood vessel. After the stent 200 has been positioned at an appropriate site, the limiting member 12 is entirely withdrawn from the hollowing portion, and then the stent 200 further expands radially without the restriction of the limiting member 12 and conforms tightly to the inner wall of the vessel.

Further, with reference to FIG. 4, there is a retraction space 41 between the other end of the outer sheath 4 (a distal end of the outer sheath 4) and the releasing assembly 2. When the outer sheath 4 moves away from the fixing member 11, the other end of the outer sheath 4 (the distal end of the outer sheath 4) gradually extends into the retraction space 41. In other words, the retraction space 41 forms an avoiding space. When the outer sheath 4 moves away from the fixing member 11, the retraction space 41 prevents the other end of the outer sheath 4 (the distal end of the outer sheath 4) from abutting against the releasing assembly 2 which would result in the failure of retraction of the outer sheath 4.

In a possible embodiment, with reference to FIG. 8, the stent delivery device 100 further includes an outer sheath sliding assembly 5, which is arranged between the fixing assembly 1 and the releasing assembly 2. The outer sheath sliding assembly 5 is mounted around the outer periphery of the outer sheath 4. The outer sheath sliding assembly 5 is configured to drive the outer sheath 4 to move away from the fixing member 11.

It should be understood that when the stent 200 is arranged within the vessel of a subject to be treated, the outer sheath sliding assembly 5 and the releasing assembly 2 are arranged outside the subject to be treated, so that the operator may control the expansion and release of the stent 200.

The outer sheath sliding assembly 5 is provided to control the outer sheath 4 to move away from the fixing member 11, which is convenient for operation and excellent in controllability.

In a possible embodiment, with reference to FIG. 8, the outer sheath sliding assembly 5 includes a second sleeve 51 and a driving assembly 52 threadedly connected to the second sleeve 51. The second sleeve 51 is mounted around the outer periphery of the outer sheath 4 and slidably connected to the outer sheath 4. The driving assembly 52 is mounted around the outer periphery of the outer sheath 4 and fixedly connected to the outer sheath 4. When the driving assembly 52 rotates relative to the second sleeve 51, the driving assembly 52 gradually moves away from the second sleeve 51, and the outer sheath 4 gradually moves away from the fixing member 11 under the action of the driving assembly 52, such that the proximal end of the stent 200 is gradually exposed from the outer sheath 4.

In a possible embodiment, with reference to FIG. 8, the driving assembly 52 includes a locking element 521 and a sliding sleeve 522. One end of the locking element 521 is threadedly connected to the second sleeve 51. The other end of the locking element 521 is detachably connected to the sliding sleeve 522. The other end of the locking element 521 may be snap-fit with the sliding sleeve 522. The locking element 521 is slidably connected to the outer sheath 4. The sliding sleeve 522 is fixedly connected to the outer sheath 4. When the locking element 521 is disengaged from the sliding sleeve 522, the sliding sleeve 522 can drive the outer sheath 4 to gradually move away from the fixing member 11.

By providing the locking element 521 and the sliding sleeve 522, with the locking element 521 being threadedly connected to the second sleeve 51, the locking element 521 is controlled to rotate relative to the second sleeve 51, so that the outer sheath 4 slowly retracts for a short distance, and the proximal end of the stent 200 gradually expands, while the locking element 521 is engaged with the sliding sleeve 522. When the locking element 521 is disengaged from the sliding sleeve 522, the sliding sleeve 522 is capable of driving the outer sheath 4 to retract rapidly for a longer distance after the restraint of the locking element 521 is removed, to allow the proximal end and the distal end of the stent 200 to expand.

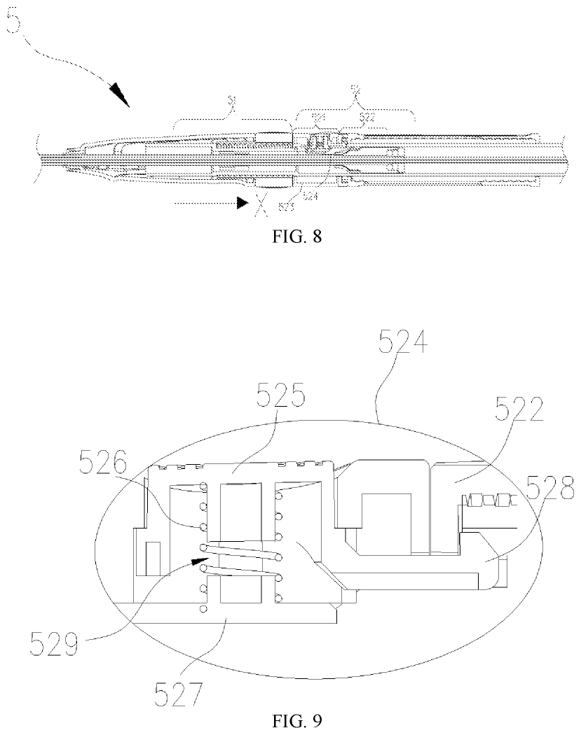

Further, with reference to FIG. 8 to FIG. 9, the locking element 521 includes a third sleeve 523 and a switch 524 arranged on the third sleeve 523. The third sleeve 523 is mounted around the outer sheath 4 and slidable relative to the outer sheath 4. The switch 524 is connected to the third sleeve 523 and slidable relative thereto in a radial direction of the third sleeve 523. A buckling portion 528 of the switch 524 is snap-fit with the sliding sleeve 522. When the switch 524 slides to a pressed position, the buckling portion 528 of the switch 524 is disengaged from the sliding sleeve 522.

Further, the switch 524 is provided with a pressing portion 525, an elastic member 526 and a base 527 which are arranged along the radial direction of the third sleeve 523 in sequence. The pressing portion 525 is arranged on an outer peripheral surface of the third sleeve 523. The elastic member 526 elastically abuts between the pressing portion 525 and the base 521, so that a gap 529 is defined between the pressing portion 525 and the base 527. The buckling portion 528 extends from one end of the pressing portion 525 towards the sliding sleeve 522. When the pressing portion 525 abuts against the base 527, the switch 524 is in the pressed position. When the sliding sleeve 522 abuts against the third sleeve 523 and the switch 524 rebounds from the pressed position under the action of the elastic member 526, the buckling portion 528 of the switch 524 is engaged with the sliding sleeve 522 under the action of the pressing portion 525.

The stent delivery device 100 according to the present application can be operated with the following process.

During the operation, firstly, a vessel of a subject to be treated is punctured, a guide wire is inserted along an extension direction of the vessel, and then the stent delivery device 100 is delivered into the aorta over the guide wire. Under X-ray fluoroscopy monitoring, the stent delivery device 100 is advanced to the vicinity of a lesion site of the vessel, the second sleeve 51 is fixed, the locking element 521 is rotated about an axial axis, and the locking element 521 together with the sliding sleeve 522 moves away from the second sleeve 51 since the locking element 521 is threadedly connected to the second sleeve 51, and thus the sliding sleeve 522 drives the outer sheath 4 to slowly retract, so that the proximal end of the stent 200 slowly expands, but the stent 200 does not fully expand yet since the proximal end of the stent 200 is still radially restrained by the limiting member 12, and thus the stent 200 stops to expand until it expands to a certain degree. Therefore, it is convenient to adjust the position of the stent 200 in the vessel at that time because the stent 200 is not completely expanded. The stent 200 is moved to the most appropriate releasing position by moving the stent delivery device 100. The switch 524 on the locking element 521 is pressed, and the buckling portion 528 is disengaged from the sliding sleeve 522. In the meanwhile, the connection between the locking element 521 and the sliding sleeve 522 are released and the sliding sleeve 522 is able to drive the limiting member 12 to move axially towards the distal end. A main body of the stent 200 expands completely. The locking member 22 is toggled to the second sliding groove 212, and the movable member 21 is pulled backwards to make the limiting member 12 move away from the fixing member 11 and further release the proximal end of the stent 200. The stent 200 is completely released from the stent delivery device 100, and the releasing process is completed. At the end, the stent delivery device 100 is withdrawn from the subject to be treated along the guide wire.

It should be understood that “pull backwards” and “retract” mentioned in the present application refer to a movement towards the operating end of the stent delivery device 100.

The above are a part of the embodiments of the present application. It should be noted that for those ordinarily skilled in the art, several improvements and modifications may be made without departing from the principle of the present application, and these improvements and modifications are regarded to be within the protective scope of the present application. It should be understood that different embodiments in the present application may be combined with each other.

What is claimed is:

1. A stent delivery device for releasing a stent into a vessel, wherein the stent delivery device comprises:
a fixing assembly, comprising a fixing member and a limiting member, wherein one end of the limiting member passes through the stent and abuts against the fixing member to lock the stent; and
a releasing assembly, comprising a movable member and a locking member connected to the movable member, wherein the movable member is connected to an other end of the limiting member, the locking member is slidably connected to the movable member, when the locking member is in a first position, the movable member is fixed relative to the fixing member under the restriction of the locking member, and when the locking member slides to a second position, the movable member is capable of driving the limiting member to move away from the fixing member so as to release the stent.

2. The stent delivery device according to claim 1, wherein the releasing assembly is provided with a first sleeve, an outer surface of the first sleeve is provided with a first mark, and the locking member is slidably connected to the first sleeve; when the locking member is located at the first position, the locking member is aligned with the first mark, and the limiting member locks the stent; and when the locking member is located at the second position, the locking member is offset from the first mark, and the limiting member releases the stent.

3. The stent delivery device according to claim 2, wherein the outer surface of the first sleeve is further provided with a second mark; and when the locking member is located at the second position, the locking member is aligned with the second mark.

4. The stent delivery device according to claim 1 wherein the movable member has a central axis, and the fixing member, the limiting member and the movable member are arranged along a direction of the central axis.

5. The stent delivery device according to claim 4, wherein an outer peripheral surface of the movable member is provided with a first sliding groove and a second sliding groove communicating each other, the first sliding groove extends along a circumferential direction of the movable member, the second sliding groove extends along the direction of the central axis, and one end of the locking member is capable of sliding in the first sliding groove and the second sliding groove, and
wherein the first position and the second position are located at the first sliding groove and the second sliding groove, respectively.

6. The stent delivery device according to claim 5, wherein the movable member is provided with a first end surface, and the second sliding groove extends through the first end surface; and wherein when the movable member moves away from the fixing member, the locking member is capable of sliding out of the second sliding groove until the locking member is disengaged from the movable member.

7. The stent delivery device according to claim 1, wherein the releasing assembly further comprises a first sleeve, the first sleeve is mounted around an outer periphery of the movable member, the first sleeve is provided with a limiting hole, the locking member is arranged on the first sleeve, and the locking member is slidably connected to the movable member through the limiting hole.

8. The stent delivery device according to claim 7, wherein the limiting hole extends along a circumferential direction of the first sleeve, the locking member is capable of sliding from one end of the first sliding groove to an other end of the first sliding groove along the limiting hole, and an inner wall of the limiting hole restricts the locking member to be fixed relative to the first sleeve in direction of the central axis.

9. The stent delivery device according to claim 7, wherein the locking member comprises a toggle portion and a sliding portion connected to each other, the toggle portion abuts against an outer peripheral surface of the first sleeve, the sliding portion passes through the limiting hole and abuts against an inner peripheral surface of the first sleeve, and the toggle portion is configured to slide relative to the first sleeve under an external force, so as to drive the sliding portion to slide along the limiting hole of the first sleeve,
wherein the toggle portion and the sliding portion are detachably connected, and
wherein the toggle portion comprises a cover plate and a hook arranged on the cover plate, the cover plate abuts against an outer surface of the first sleeve, the sliding portion is provided with a first sliding portion and a second sliding portion connected to each other, with a through hole extending through the first sliding portion and the second sliding portion, the first sliding portion is slidably engaged in the limiting hole of the first sleeve, and the second sliding portion is slidably engaged in the first sliding groove and the second sliding groove of the movable member; and the hook is engaged with the second sliding portion through the through hole, so that one end of the first sliding portion abuts against the cover plate.

10. The stent delivery device according to claim 1, wherein the fixing assembly further comprises a guide member, the guide member is arranged opposite to the fixing member with an accommodating space defined between the guide member and the fixing member, and the accommodating space is configured to accommodating an end portion of the stent, and wherein one end of the limiting member passes through the guide member and extends into the accommodating space, and passes through the end portion of the stent within the accommodating space and is connected to the fixing member.

11. The stent delivery device according to claim 10, wherein the fixing member is provided with at least one first hole facing the guiding member; and when the locking member is in the first position or the second position, one end of the limiting member extends into or exits from the first hole when being driven by the movable member,
wherein the guide member is provided with at least one second hole, and the second hole is aligned with the first hole, and the limiting member passes through the second hole and is slidably connected to the guide member, and
wherein when the limiting member moves away from the fixing member when being driven by the movable member, an end portion of the limiting member is capable of retracting into the second hole, and the end portion of the stent is disengaged from the limiting member and is capable of expands radially when the end portion of the limiting member is retracted into the second hole.

12. The stent delivery device according to claim 1, wherein the stent delivery device further comprises an inner core, the fixing member and the movable member are mounted around an outer periphery of the inner core, the fixing member is fixed relative to the inner core, and the movable member is slidably connected to the inner core.

13. The stent delivery device according to claim 12, wherein the stent delivery device further comprises an outer sheath, wherein the outer sheath is mounted around the outer periphery of the inner core, an accommodating cavity is defined between the outer sheath and the inner core, and the stent is compressed within the accommodating cavity.

14. The stent delivery device according to claim 13, wherein the inner core and the outer sheath are made of flexible material, wherein the limiting member is arranged within the accommodating cavity, and the stent is arranged between the limiting member and the outer sheath, and
wherein the limiting member is made of flexible material.

15. The stent delivery device according to claim 13, wherein one end of the outer sheath abuts against the fixing member in an initial state, the outer sheath is capable of sliding relative to the inner core; and wherein when the outer sheath moves away from the fixing member, a portion of the stent which exposes from the outer sheath expands under an action of its own tension.

16. The stent delivery device according to claim 15, wherein a retraction space is provided between an other end of the outer sheath and the releasing assembly; and wherein when the outer sheath moves away from the fixing member, the other end of the outer sheath extends into the retraction space.

17. The stent delivery device according to claim 13, wherein the stent delivery device further comprises an outer sheath sliding assembly, the outer sheath sliding assembly is arranged between the fixing assembly and the releasing assembly, the outer sheath sliding assembly is mounted around an outer periphery of the outer sheath, and the outer sheath sliding assembly is configured to drive the outer sheath to move away from or close to the fixing member, and
wherein the outer sheath sliding assembly comprises a second sleeve and a driving assembly threadedly connected to the second sleeve, and the second sleeve is mounted around the outer periphery of the outer sheath and slidably connected to the outer sheath; the driving assembly is mounted around the outer periphery of the outer sheath and fixedly connected to the outer sheath; and wherein when the driving assembly rotates relative to the second sleeve, the driving assembly gradually moves away from the second sleeve, and the outer sheath driven by the driving assembly gradually moves away from the fixing member.

18. The stent delivery device according to claim 17, wherein the driving assembly comprises a locking element and a sliding sleeve, one end of the locking element is threadedly connected to the second sleeve, the locking element is slidably connected to the outer sheath, and an other end of the locking element is detachably connected to the sliding sleeve; and the sliding sleeve is fixedly connected to the outer sheath, and wherein when the locking member is disengaged from the sliding sleeve, the sliding sleeve is capable of driving the outer sheath to gradually move away from the fixing member.

19. The stent delivery device according to claim 18, wherein the locking member comprises a third sleeve and a switch provided on the third sleeve, the third sleeve is mounted around the outer sheath and slidable relative to the outer sheath, the switch is slidably connected to the third sleeve along a radial direction of the third sleeve, and the switch has a buckling portion engaged with the sliding sleeve; and wherein when the switch slides to a pressed position, the buckling portion of the switch is disengaged from the sliding sleeve.

20. The stent delivery device according to claim 19, wherein the switch is provided with a pressing portion, an elastic member and a base arranged in sequence along a radial direction of the third sleeve, the pressing portion is arranged on an outer peripheral surface of the third sleeve, the elastic member elastically abuts between the pressing portion and the base with a gap defined between the pressing portion and the base; the buckling portion extends towards the sliding sleeve from one end of the pressing portion; and wherein when the pressing portion abuts against the base, the switch is in the pressed position; while when the sliding sleeve abuts against the third sleeve and the switch rebounds from the pressed position under an action of the elastic member, the buckling portion of the switch is engaged with the sliding sleeve under an action of the pressing portion.
